SEQUENCE LISTING (8 sequences)

SEQ ID NO: 1 - DNA, 20 bp, Artificial sequence
  Synthetic Oligonucleotides
acggcatggatctcaaagac

SEQ ID NO: 2 - DNA, 20 bp, Artificial sequence
  Synthetic Oligonucleotides
gtgggtgaggagcacgtagt

SEQ ID NO: 3 - DNA, 20 bp, Artificial sequence
  Synthetic Oligonucleotides
ttaaggttctctcctctgaa

SEQ ID NO: 4 - DNA, 20 bp, Artificial sequence
  Synthetic Oligonucleotides
tagggagctaaattatccaa

SEQ ID NO: 5 - DNA, 20 bp, Artificial sequence
  Synthetic Oligonucleotides
ggttgccaagccttatcgga

SEQ ID NO: 6 - DNA, 19 bp, Artificial sequence
  Synthetic Oligonucleotides
acctgctccactgcttgct

SEQ ID NO: 7 - DNA, 24 bp, Artificial sequence
  Synthetic Oligonucleotides
gcaatagttccttcccagagttct

SEQ ID NO: 8 - DNA, 19 bp, Artificial sequence
  Synthetic Oligonucleotides
ggatggcccatcggataag

(19) United States
(12) Patent Application Publication
(10) Pub. No.: US 2010/0275282 A1
(43) Pub. Date: Oct. 28, 2010

(54) METHODS AND SYSTEMS FOR IDENTIFYING IMMUNOMODULATORY SUBSTANCES

(76) Inventors: JUNE L. ROUND, PASADENA, CA (US); YUE SHEN, PASADENA, CA (US); SARKIS K. MAZMANIAN, PORTER RANCH, CA (US)

Correspondence Address: Steinfl & Bruno, 301 N Lake Ave Ste 810, Pasadena, CA, 91101, US

(21) Appl. No.: 12/766,787

(22) Filed: Apr. 23, 2010

Related U.S. Application Data
(60) Provisional application No. 61/172,101, filed on Apr. 23, 2009.

(51) Int. Cl. A01K 67/00; C12Q 1/02; C12Q 1/37; A61K 35/74

(52) U.S. Cl. 800/13; 435/29; 435/23; 424/93.4

(57) ABSTRACT

A method and system is presented for screening bacteria, products purified or made by bacteria and/or other bacterial substance for anti-inflammatory ability.

### CROSS REFERENCE TO RELATED APPLICATIONS

This application claims priority to U.S. Provisional Application No. 61/172,101, entitled “High-throughput Screen for Bacteria with Anti-inflammatory Properties”, filed on Apr. 23, 2009, Docket No. CIT-5354-P, the disclosure of which is incorporated herein by reference in its entirety.

### STATEMENT OF GOVERNMENT GRANT

The U.S. Government has certain rights in this invention pursuant to Grant No. DK078938 awarded by the National Institutes of Health.

### FIELD

The present disclosure relates to methods and systems for identifying immunomodulatory substance and in particular immunomodulatory microorganisms and compounds.

### BACKGROUND

Identification of immunomodulatory microorganisms and compounds is of particular interest. The human gastrointestinal tract harbors an extraordinary number of microbes (known as the gut microbiota) that have a profound effect on the development and function of the immune system. Of the innumerable species of bacteria that inhabit the gastrointestinal tract of mammals, Bacteroidetes are the most abundant Gram-negative bacterial phylum.

In particular, Bacteroides fragilis is a human commensal microorganism that has been shown to have immunomodulatory properties. In particular, immunomodulatory properties of Bacteroides fragilis have been associated to Polysaccharide A (PSA) production by the bacterium.

However, identification of the mechanism and/or molecular triggers by which B. fragilis (or any commensal bacteria) delivers beneficial microbial molecules to the immune system and performs the immunomodulatory properties has been challenging.

### SUMMARY

Provided herein, are methods and systems for screening microorganisms and related substances having immunomodulatory and, in particular, anti inflammatory properties. In particular, provided herein are methods and systems that in several embodiments allow identification of bacterial substances that are able to induce in an individual an immunomodulatory response comparable to the one of Bacteroides fragilis.

According to a first aspect, a method is described for identifying a bacterial substance having immunomodulatory ability. The method comprises contacting a candidate bacterial substance with a T cell alone or in presence of an antigen presenting cell, and detecting expression of at least one of one or more anti inflammatory biomarkers selected from the group consisting of IL-10, Foxp3, TGFβ1, TGFβ2 Perforin and Granzyme B, and one or more inflammatory biomarkers selected from the group consisting of IFNγ, IFNα, IFNβ IL-1β, IL-4, IL-5, IL-6, IL-8, IL-9, IL-13, IL-21, IL-22, IL-23, IL-17 or TNFα. The method further comprises determining an anti inflammatory ability of the candidate bacterial substance through detection of an increase of the expression of the one or more anti-inflammatory biomarkers or a decrease of the expression of the one or more inflammatory biomarkers following the contacting.

According to a second aspect a method is described for identifying a bacterial substance having immunomodulatory ability. The method comprises contacting a candidate bacterial substance with an antigen presenting cell, and incubating the antigen presenting cell with a T cell following the contacting. The method further comprises detecting expression of at least one of one or more anti inflammatory biomarker selected from the group consisting of IL-10, Foxp3, TGFβ1, TGFβ2, Perform and Granzyme B, and one or more inflammatory biomarker selected from the group consisting of IFNγ, IFNα, IFNβ IL-β, IL-4, IL-5, IL-6, IL-8, IL-9, IL-13, IL-21, IL-22, IL-23, IL-17 or TNFα. The method further comprises determining an anti inflammatory ability of the candidate bacterial substance through detection of an increase of the expression of the one or more anti-inflammatory biomarkers or a decrease of the expression of the one or more inflammatory biomarkers following the incubating.

According to another aspect, a method is described for identifying a bacterial substance having immunomodulatory ability in animals. The method comprises treating a transgenic marker non-human animal with a candidate bacterial substance, the transgenic marker non-human animal genetically modified to express at least one of one or more labeled inflammatory biomarkers selected from the group consisting of IL-10, Foxp3, TGFβ1, TGFβ2, Perforin and Granzyme B, and one or more labeled inflammatory biomarker selected from the group consisting of IFNγ, IFNα, IFNβ IL-1β, IL-4, IL-5, IL-6, IL-8, IL-9, IL-13, IL-21, IL-22, IL-23, IL-17 or TNFα. The method further comprises detecting expression in the transgenic marker non-human animal of at least one of the one or more anti-inflammatory biomarkers or at least one of the inflammatory biomarkers following the treating and determining an anti inflammatory ability of the candidate bacterial substance through detection of an increase of the expression of the one or more anti-inflammatory biomarkers or a decrease of the expression of the one or more inflammatory biomarkers following the treating.

According to another aspect a system for screening a bacterial substance is described. The system comprises at least two of a T cell, an antigen presenting cell and reagents for detection of at least one of one or more anti-inflammatory biomarkers selected from the group consisting of IL-10, Foxp3, TGFβ1, TGFβ2, Perform and Granzyme B and one or more labeled inflammatory biomarker selected from the group consisting of IFNγ, IFNα, IFNβ IL-1β, IL-4, IL-5, IL-6, IL-8, IL-9, IL-13, IL-21, IL-22, IL-23, IL-17 or TNFα, for simultaneous, combined or sequential use in a method to identify an anti-inflammatory bacterial substance herein described.

The methods and systems herein described can be used in connection with medical, pharmaceutical, veterinary applications as well as fundamental biological studies and various applications, identifiable by a skilled person upon reading of the present disclosure, wherein investigating immunomodulatory ability and in particular anti-inflammatory ability of a substance is desirable

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows that outer membrane vesicles from Bacteroides fragilis contain PSA. FIG. 1a shows OMV produced wild type B. fragilis (WT-OMV) and B. fragilis ΔPSA (ΔPSA-OMV) detected by transmission electron microscopy of EDL (electron dense layer) enriched B. fragilis. FIG. 1b shows an immunoblot analysis of whole cell (WC) and outer membrane vesicles (OMV) extracts from wild-type and PSA-mutant bacteria. FIG. 1c shows immunogold labeling of purified OMVs, stained with anti-PSA and anti-IgG-colloidal gold conjugate (5 nm), analyzed by electron microscopy. FIG. 1d shows a glycoprotein staining of capsular polysaccharide preparations from whole cells and OMVs.

FIG. 2 illustrates exemplary results showing that OMVs protect animals from experimental colitis and intestinal inflammation in a PSA-dependent manner. FIG. 2a shows a diagram reporting weight loss in animal groups following the induction of TNBS colitis (day 0) measured as reduction from initial weight until day of sacrifice (day 4). All groups contained at least 4 animals, with error bars indicating standard error of the mean (SEM). Results are representative of 3 independent trials. p values determined by one-way ANOVA: * p<0.05; *** p<0.001. FIG. 2b shows images of unmanipulated colons immediately following resection and quantification of length (graph) from vehicle treated (EtOH) and TNBS groups (n=4 animals/group). Error bars indicate SEM. Results are shown from 3 combined experiments performed independently. p values determined by one-way ANOVA: *** p<0.001. NS: not significant. FIG. 2c shows images from hematoxylin and eosin (H & E) stained colon sections representative of each treatment group. FIG. 2d shows colitis scores from animals assigned by a blinded pathologist (G.W.L) according to a standard scoring system (ONLINE METHODS) (Scheiffele and Fuss. (2001) Induction of TNBS colitis in mice. Current Protocols in Immunology. 1.19.1-15.19.14) Each symbol represents an individual animal. Results are shown from 3 combined experiments performed independently. *** p<0.001. NS: not significant. FIGS. 2e and f show diagrams illustrating cytokine transcript analysis by qRT-PCR from RNA recovered from whole colons (FIG. 2e) or purified CD4⁺ T cell from mesenteric lymph nodes (FIG. 2f). Error bars indicate SEM from 4 animals/group. Results are representative of 3 independent trials.

FIG. 3 shows results indicating that PSA containing OMVs induce IL-10 production and Foxp3 expression from T cells co-cultured with treated DCs. FIG. 3a shows flow cytometry (FC) analysis of OMV internalization by DCs. OMVs were labeled with FITC (Fluorescein isothiocyanate) and incubated with cultured DCs for various times (as indicated). Cells were stained with anti-CD11c. Percentages show CD11C⁺OMV⁺ populations. FIG. 3b shows FC plots of DCs incubated with WT-OMVs and ΔPSA-OMVs for various times (as indicated) and stained with anti-CD11c and anti-MHCII. Percentages show MHCII⁺ populations among CD11c⁺ cells. FIG. 3c shows ELISA analysis for IL-10 of culture supernatants from DCs or DC-T cell co-cultures, where DCs were pulsed with OMVs for 18 hours, washed and incubated with primary CD4⁺T cells or not. Supernatants were collected at day 4 of culture. Media samples indicate DCs that were not pulsed with OMVs, but otherwise treated identically. Anti-CD3 was added to some samples to augment T cell responses. Error bars indicate SEM from triplicate samples. Results are representative of over 5 independent trials. * p<0.05; ** p<0.01. FIG. 3d (left panel) shows ELISA analysis similar to FIG. 3c, but also including DCs differentiated from IL-10^{-/-}animals. Error bars indicate SEM from triplicate samples. Results are representative of 3 independent trials. * p<0.05. NS: not significant. FIG. 3d (right panel) shows ELISA analysis similar to FIG. 3c, but also including DCs differentiated from TLR2^{-/-}animals. SEA: staphylococcal enterotoxin A. Error bars indicate SEM from triplicate samples. Results are representative of 3 independent trials. * p<0.05. NS: not significant. FIG. 3e shows transcript levels of IL-10 (left) and Foxp3 (right) as determined by qRT-PCR of RNA received from purified T cell subsets following in vitro culture with DCs. Co-cultures were set up as in (c-e); on day 4, CD4⁺CD25⁺ and CD4⁺CD25⁻T cell were purified by magnetic bead separation (>95% purity) and RNA extracted with RNeasy mini kit. Relative values were normalized to β-actin. Error bars indicate SEM from triplicate samples. Results are representative of 3 independent trials. * p<0.05; *** p<0.001. NS: not significant. FIG. 3f shows co-cultures were set up as in (c-e), but using CD4⁺T cells from Foxp3-GFP mice. Following 4 days of culture with OMV pulsed DCs, cells were stained with anti-CD4 and Foxp3 detected by GFP expression using FC. Results are representative of 2 independent trials.

FIG. 4 shows exemplary results indicating in vitro generation of Treg suppressive function by PSA-containing OMVs. FIG. 4a shows FC histograms of IL-10 expression by CD4⁺T cell subsets following 4 day co-culture with DCs treated with OMVs. Splenic CD4⁺T cell were purified from IL-10-GFP mice, stained with anti-CD4 and anti-CD25 following co-culture, and IL-10 expression measured by GFP expression. Percentages show IL-10⁺ populations among CD4⁺CD25⁺ and CD4⁺CD25⁻subsets. Results are representative of 3 independent trials. FIG. 4b shows in vitro suppression of naïve responder cells by purified CD4⁺CD25⁺T cells following co-culture with DCs treated with media (control), WT-OMVs and ΔPSA-OMVs. Cell proliferation was measured by FC of CFSE dilution. Treg:Teff ratios are indicated, and percentages show total proliferating cells. No Treg: CD4⁺CD25⁻cells only. Results are representative of 2 independent trials. FIG. 4c shows the quantification of percentage of CD4⁺T cells in each proliferating peak (as is labeled as 1, 2, 3, 4, 5 in FIG. 4b). Results are shown from 3 combined experiments performed independently. * p<0.05. NS: not significant.

FIG. 5 shows wild-type B. fragilis and PSA deletional mutant B. fragilis produce similar amount of OMVs during in vitro culture. The amount of total protein recovered from each OMVs preparation normalized by OD₆₀₀ of the culture at the time of harvest. Error bars indicate SEM. Result is shown from >10 combined experiments preformed independently. p value determined by Student's t-test. NS: not significant.

FIG. 6 shows Majority OMVs purified from wild-type B. fragilis contain PSA Immunogold labeling of PSA on purified OMVs shows ˜60% of WT-OMVs observe among random sampling of 10 areas (1 μm×1 μm) of sample are associated with PSA, but none of ΔPSA-OMVs observed are stained positively for PSA. (Immunogold labeling of WT-OMvs with anti-IgG-gold only confirms the specificity of the labeling).

FIG. 7 shows OMVs from wild-type or PSA deletional mutant B. fragilis show no significant difference in protein composition. Proteome mass spectrometry shows 100% overlap of the identified proteins (>1 unique peptide identified for each protein) between WT-OMVs and ΔPSA-OMVs. Among all of the identified proteins, we semi-quantitatively compared the amount of those relatively abundant proteins according to the number of unique peptides identified for each of them. Majority of them show no difference performed independently. Errors indicate SEM. p value determined by Student's t-test. **: p<0.01; *: p<0.05; NS: not significant.

FIG. 8 shows actin polymerization is required for OMV uptake by DCs. Flow cytometry analysis of OMV internalization by DCs pre-treated with Cytochlasin D. OMVs were labeled with FITC (Fluorescein isothiocyanate) and incubated with cultured DCs for various times (as indicated). Cells were stained with anti-CD11c. Percentages show CD11C⁺OMV⁺ populations.

FIG. 9 shows WT-OMVs or ΔPSA-OMVs are internalized and localized in the cytoplasm of DCs. Fluorescent micrographs of OMV (WT or ΔPSA) internalization by DCs. OMVs were labeled with FITC (gray arrow) and incubated with cultured DCs for 2 hrs. Cells were fixed and cell membrane was stained with Wheat Germ Agglutinin (WGA)-tetramethylrhodamine (black arrow). Scale bar: 7.5 μm.

FIG. 10 shows WT-OMVs and ΔPSA-OMVs upregulate co-stimulatory molecule for DC activation. FC plots of DCs incubated with WT-OMVs and ΔPSA-OMVs for various times (as indicated) and stained with anti-CD11c and anti-CD86. Percentages show CD86⁺ populations among CD11c⁺ cells.

FIG. 11 shows PSA induced expression of biomarkers in an embodiment herein described. The data illustrated in each diagram are representative of three independent experiments. Light bars indicated cells derived from PBS treated mice and dark bars from PSA-treated mice.

### DETAILED DESCRIPTION

Methods and systems are described that are suitable for identifying an immunomodulatory bacterial substance.

The term “immunomodulatory” as used herein indicates the ability to promote a state associated with absence of an inflammatory response. Particular immunomodulatory properties comprise anti inflammatory properties, wherein the term anti-inflammatory refers to the property of a substance or treatment that prevents or reduces inflammation.

The term “inflammation”, “inflammatory state” or “inflammatory response” as used herein indicate the complex biological response of vascular tissues of an individual to harmful stimuli, such as pathogens, damaged cells, or irritants, and includes secretion of cytokines and more particularly of pro-inflammatory cytokine, i.e. cytokines which are produced predominantly by activated immune cells such as microglia and are involved in the amplification of inflammatory reactions. Exemplary pro-inflammatory cytokines include but are not limited to IL-1, IL-6, TNF-a, IL-17, IL21, and IL23, -. Exemplary inflammations include acute inflammation and chronic inflammation. The wording “acute inflammation” as used herein indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). The wording “chronic inflammation” as used herein indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis.

The term “substance” as used herein indicates a matter of particular or definite chemical constitution. The term “bacterial substance” indicate matter of bacterial origin such as, a live bacteria, dead bacteria, and in particular heat-killed bacteria, bacterial extracts, purified molecules from bacteria, a combination of molecules purified from bacteria, or vesicles containing one molecule or a combination of molecules purified from bacteria or purified vesicles from bacteria. In particular, bacterial substance can be formed by outer membrane vesicles (OMVs) which are vesicles released from the surface of bacteria and deliver a suite of molecular cargo to distant target cells. Bacterial substance comprises substance derived from a same bacteria and substance derived from two or more different bacteria.

The term “bacteria” as used herein indicates large group of unicellular, prokaryote, microorganisms, typically of a few micrometers in length, and having a wide range of shapes. In particular, bacteria in the sense of the present disclosure comprise bacteria of the human flora, i.e. the assemblage of microorganisms that reside on the surface and in human tissues and bodily fluids such as deep layers of skin, in saliva, oral or vaginal mucosa, and in the gastrointestinal tracts. Bacterial flora comprises gut flora (bacteria detectable in the digestive tract of humans) vaginal flora (bacteria detectable in the fibromuscular tubular tract leading from the uterus to the exterior of the body in female humans) and skin flora (bacteria detectable in human skin). More specifically, bacteria in the sense of the present disclosure can be formed by one or more bacteroidetes of the gut flora and in particular one or more bacteroides, a genus of Gram-negative, non endospore forming anaerobes bacillus bacteria, symbiotic with humans and identifiable by a skilled person. A representative bacteroides is B. fragilis.

In methods and systems herein described, the immunomodulatory ability of a bacterial substance can be determined by detection of inflammatory and/or anti-inflammatory biomarkers. The term “biomarker” as used herein indicates a substance or characteristic used as an indicator of a biological state, such as a phase of cellular cycle, a biological process, as well as health and disease state. The presence, absence, reduction, upregulation of the biomarker is associated with and is indicative of a particular state. In particular, the term “inflammatory biomarker” as used herein indicates a biomarker that is indicative of presence of an inflammatory state and comprise an IFNγ, IFNα, IFNβ IL-1β, IL-4, IL-5, IL-6, IL-8, IL-9, IL-13, IL-21, IL-22, IL-23, IL-17, TNFα and additional cell markers and cytokines identifiable by a skilled person. The term anti-inflammatory biomarker as used herein indicates a biomarker that is indicative of absence of an inflammatory state and comprises Foxp3, IL-10, TGFβ1, TGFβ2 Perforin and Granzyme B and additional cell markers and cytokines identifiable by a skilled person.

The term “transgenic marker non-human animal” as used herein refers to an animal that contains non-native, genetic material that has been transferred naturally or by any of a number of genetic engineering techniques. Such non-native, genetic material (or transgene) may act as a “biomarker” (as defined herein) and/or retain the ability to produce RNA or protein in the non-human animal.

In an embodiment, identification of an immunomodulatory bacterial substance is performed by detecting expression of inflammatory or anti-inflammatory biomarkers by a T cells alone or in presence of an antigen presenting cell.

The term “T cell” as used herein indicates a indicates a sub-group of lymphocytes (a type of white blood cell or leukocyte) including different cell types identifiable by a skilled person. T-helper cell according to the present disclosure and include effector Tₕ cells (such as Th1, Th2 and Th17)—i.e. Th cells that secrete cytokines, proteins or peptides that stimulate or interact with other leukocytes, including Tₕ cells—and suppressor Th cells (such as Treg) i.e. Th cells that suppress activation of the immune system and thereby maintain immune system homeostasis and tolerance to self-antigens.

The term “antigen presenting cell” as used herein indicates a cell that displays foreign antigen complex with major histocompatibility complex (MHC) on its surface. In particular, antigen presenting cell comprise dendritic cell, macrophage, B cells and additional cells identifiable by a skilled person.

In an embodiment, methods herein described comprise contacting a candidate bacterial substance with a T cell alone or in presence of an antigen presenting cell. In an embodiment, methods here described comprise contacting a candidate bacterial substance with an antigen presenting cell and incubating the antigen presenting cell with a Tcell following the contacting.

The term “contacting” or “incubating” as used herein indicates actions directed to creation of a spatial relationship between two items provided for a time and under condition such that at least one of the reciprocal or non reciprocal action or influence between the two items can be exerted. In particular, incubation can be performed between a bacterial substance and a cell and can result in a direct contact and/or interaction between the bacterial substance and the cell or can result in a modification of the cell following an indirect action of the bacterial substance (e.g. following activation or modification of another substance which directly interacts with the cell).

Incubation can also be performed between a first cell and a second cell following contacting of the first cell with a bacterial substance and can result in a direct contact and/or interaction between the first cell and the second cell or can result in a modification of the second cell following an indirect action of the first cell (e.g. following secretion of cytokines or other molecules which directly interact with the second cell).

Exemplary contacting a bacterial substance with a T cell or an antigen presenting cell can be performed in vitro by bathing a whole sample comprising one or more types of cells, in a solution containing the bacterial substance under suitable conditions which depend on the specific cells and the specific bacterial substance and are identifiable by a skilled person upon reading of the present disclosure. Additionally exemplary contacting between the bacterial substance and a T cell or antigen presenting cell can be performed in vitro by introducing the bacterial substance to a cell culture of purified cells under suitable conditions, and in vivo by treating an individual (e.g. a transgenic animal, other than a human, genetically modified to express a labeled inflammatory or an anti inflammatory biomarker) with the bacterial substance. The treating can be performed by administering the bacterial substance by topical or systemic administration. Systemic administration includes enteral administration (e.g. oral administration, administration by gastric feeding tube, administration by duodenal feeding tube, gastrostomy, enteral nutrition, and rectal administration) and parenteral administration (e.g. intravenous administration, intra-arterial administration, intramuscular administration, subcutaneous administration, intradermal, administration, intraperitoneal administration, and intravesical infusion. Topical administration include but is not limited to epicutaneous administration, inhalational administration (e.g. in asthma medications), enema, eye drops (E.G. onto the conjunctiva), ear drops, intranasal route (e.g. decongestant nasal sprays), and vaginal administration.

Exemplary incubation of an antigen presenting cell with a T cell can be performed in vitro by mixing a cell culture comprising the antigen presenting cell with a cell culture comprising the T cell, by adding a purified antigen presenting cell to a culture of T cells, or by adding a purified T cell to a culture of antigen presenting cells. Additional exemplary incubation between a T cell and an antigen presenting cell in vivo comprise transplanting antigen presenting cell in a into a tissue of an individual, the tissue comprising T cell, or transplanting T cell into a tissue of an individual the tissue comprising antigen presenting cell. In an embodiment, the individual is a transgenic animal other than humans genetically modified to express a labeled inflammatory or an anti inflammatory biomarker.

Additional procedures and techniques suitable for performing contacting between a substance and a T cell or antigen presenting cell and incubation between an antigen presenting cell and a T cell in vitro or in vivo can be identified by a skilled person upon reading of the present disclosure

Suitable conditions for performing the contacting or incubation are identifiable by a skilled person and comprise providing a suitable environment for cell culture in vitro or suitable environment for e.g. by use of a robotic incubator which controls and monitors the temperature and optionally also the atmospheric CO₂, N₂ and/or O₂ content, relative humidity, nutrient amounts and other conditions suitable to provide the cells with a suitable growth environment. Also additional procedures and techniques suitable for performing contacting between a substance and a cell in vitro or in vivo can be identified by a skilled person upon reading of the present disclosure.

The amount of substance given to in vitro cultures or in vivo to animals, as well as the time frame of incubation can be determined by the skilled person experimentally in view of the specific bacterial substance, the specific T cell, antigen presenting cell, animal treated, related suitable conditions and the experimental design.

In methods and systems herein described, detection of the expression of an inflammatory or an anti-inflammatory biomarker can be performed in vitro and in vivo by techniques identifiable by a skilled person which comprise use of labeled molecules, including labeled biomarkers or labeled molecule specific for the biomarker or molecule associated thereto.

The term “detect” or “detection” as used herein indicates the determination of the existence, presence or fact of an analyte or related signal in a limited portion of space, including but not limited to a sample, a reaction mixture, a molecular complex and a substrate. A detection is “quantitative” when it refers, relates to, or involves the measurement of quantity or amount of the analyte or related signal (also referred as quantitation), which includes but is not limited to any analysis designed to determine the amounts or proportions of the analyte or related signal. A detection is “qualitative” when it refers, relates to, or involves identification of a quality or kind of the analyte or related signal in terms of relative abundance to another analyte or related signal, which is not quantified.

The terms “label” and “labeled molecule” or as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “signal” or “labeling signal” as used herein indicates the signal emitted from the label that allows detection of the label, including but not limited to radioactivity, fluorescence, chemiluminescence, production of a compound in outcome of an enzymatic reaction and the like.

Detection can be performed by detecting levels of expression of the biomarker, a precursor or analog thereof, and/or of an analyte associated thereto. The wording “associated to” as used herein with reference to two items indicates a relation between the two items such that the occurrence of a first item is accompanied by the occurrence of the second item, which includes but is not limited to a cause-effect relation and sign/symptoms-disease relation. In particular, the detection can be performed qualitatively or quantitatively and can involve detection of molecules such as RNA, protein, their precursors, differing types (i.e. mRNA, tRNA, and rRNA) and/or degradation products, and/or detection or measurable properties associated thereof. Techniques and procedures to perform detection are identifiable by a skilled person upon reading of the present disclosure.

Exemplary methods for detection of a biomarker expression comprise methods known to a skilled person including but not limited to ELISA, Q-PCR and intracellular cytokine staining detected by FACs. In some embodiments, expression of a biomarker can be detected via fluorescent based readouts on a cell culture performed using an antibody specific for the biomarker or molecule associated thereto, labeled with fluorophore, which includes, but not exhaustively, small molecular dyes, protein chromophores, quantum dots, and gold nanoparticles. In an embodiment expression of a biomarker can be detected by detecting expression of a label under the transcriptional control of a biomarker promoter in vivo (e.g., in an animal tissue) or in vitro (e.g. in a cell culture). In some of those embodiments the biomarker can be in particular IL-10 or Foxp3. Additional techniques are identifiable by a skilled person upon reading of the present disclosure and will not be further discussed in detail.

In methods and systems herein described an anti inflammatory ability of the candidate bacterial substance can be determined through detection of an increase of expression of the one or more anti-inflammatory biomarkers or a decrease of the expression of the one or more inflammatory biomarkers following the contacting and/or the incubating. Determining increase and/or decrease of a biomarker expression can be performed by comparing the detected expression of the biomarker following the contacting and/or incubating, with a predetermined detected expression of the same biomarker in absence of contacting and/or incubating.

Determining increase and/or decrease of a biomarker expression can be performed by comparing the detected expression marker with the detected expression of the same biomarker in a control cell in absence of contacting and/or incubating.

Reference is made to the Examples section wherein increase in expression of the anti-inflammatory biomarkers IL-10, Foxp3, TGFβ1, TGF-β2, perforin and granzyme B and decrease of expression in inflammatory biomarkers TNF-α or IL-17A are associated with the mechanism of action of Bacteroides fragilis supporting association between the above expression pattern and the biological activities of Bacteroides fragilis, with particular reference to anti-inflammatory abilities. The anti-inflammatory abilities of Bacteroides fragilis are identifiable by a skilled person and described in various publications and patent including but not limited to U.S. Pat. No. 05,679,654, U.S. Pat. No. 07,083,777, US2004092433, WO07092451 and WO2009062132, each of which is herein incorporated by reference in its entirety. A skilled person will understand that the specific anti-inflammatory ability of the candidate bacterial substance will be possibly determined based on further determination of the specific set of cytokines produced and cells activated by the candidate bacterial substance identified by methods herein described.

In an embodiment, detection of an increase in expression of the anti-inflammatory or detection of a decrease in expression of an inflammatory biomarker indicates the ability of a substance to induce anti-inflammatory molecules such as IL-10, Foxp3, TGFβ1 or TGF-β2, perforin and granzyme B or a combination thereof, or suppress inflammatory cytokines such as IFNγ, IFNα, IFNβ IL-1β, IL-4, IL-5, IL-6, IL-8, IL-9, IL-13, IL-21, IL-22, IL-23, IL-17 or TNFα or a combination thereof.

In an embodiment, the method comprises contacting a candidate bacterial substance in vitro and/or in vivo with a T cell alone or in presence of an antigen presenting cell, and detecting expression of at least one anti inflammatory biomarker selected from the group consisting of IL-10, Foxp3, TGFβ1, TGFβ2, perform and granzyme B, wherein an increase of the expression of the anti-inflammatory biomarker following the contacting indicates an anti inflammatory ability of the candidate bacterial substance. In particular, in some of those embodiments, expression of IL-10, TGFβ1, TGFβ2, perforin and/or granzyme B can be performed on Foxp3 expressing T cell, and more particularly Foxp3 expressing Treg cell.

In an embodiment, the method comprises contacting a candidate bacterial substance with an antigen presenting cell, and incubating the antigen presenting cell with a T cell following the contacting. The method further comprises detecting the expression of at least one anti-inflammatory biomarker selected from the group consisting of Foxp3, IL-10, TGFβ1, TGFβ2, TGFβ2, Perforin and Granzyme B, wherein detection of an increase of the biomarker expression following the incubating indicates an anti inflammatory ability of the candidate substance. In particular, in some of those embodiments, expression of IL-10, perforin and/or granzyme B can be performed on Foxp3 expressing T cell, and more particularly Foxp3 expressing Treg cell.

In an embodiment, the method comprises contacting a candidate bacterial substance with a T cell alone or in presence of an antigen presenting cell, and detecting expression of at least one inflammatory biomarker selected from the group consisting of IL17 and TNFα, wherein a decrease of the expression of the inflammatory biomarker following the contacting indicates an anti-inflammatory ability of the candidate bacterial substance. In particular, in some of those embodiments, expression of IL17 and TNFα can be performed on Foxp3 expressing T cell, and more particularly Foxp3 expressing Treg cell.

In an embodiment, the method comprises contacting a candidate bacterial substance with an antigen presenting cell, and incubating the antigen presenting cell with a T cell following the contacting. The method further comprises detecting the expression of at least one anti-inflammatory biomarker selected from the group consisting of consisting of IL17 and TNFα, wherein a decrease of the expression of the inflammatory biomarker following the contacting indicates an anti-inflammatory ability of the candidate bacterial substance. In particular, in some of those embodiments, expression of IL17 and TNFα can be performed on Foxp3 expressing T cell, and more particularly Foxp3 expressing Treg cell.

In an embodiment, the candidate bacterial substance comprises bacteria in a pure pool. In other embodiments, the candidate bacterial substance comprises bacteria in a mixed pool. The bacteria can be live bacteria, dead bacteria, extracts or products isolated from bacteria, or combinations of each. The bacteria may also be from laboratory strains, isolates from repositories such as ATCC, isolates from animals, isolates from humans, or combinations of each.

In an embodiment, the isolate from animals is isolated from feces. The isolate can also come from intestinal contents or combinations of feces and other intestinal contents. In one embodiment, the isolate from humans is isolated from feces. The isolate can also come from intestinal contents or combinations of feces and other intestinal contents.

In an embodiment, the method comprises treating a transgenic marker non-human animal and in particular a transgenic marker mammal such as a mouse or a rat, with a candidate substance, the transgenic non-human marker animal genetically modified to express a labeled anti-inflammatory or inflammatory biomarker selected from the group consisting of IL-10 and Foxp3 but also perform and granzyme B, IL17 and TNFα; and detecting biomarker expression in the transgenic marker animal following the treating.

In particular, in one embodiment the aforementioned transgenic non-human animal models can be treated either orally or intravenously with the substance, or permanently colonized with live bacteria by oral gavage and the amount of biomarker expression Foxp3 or IL10 expression will be monitored in the various compartments of the mouse including the spleen, mesenteric lymph nodes, small and large intestine, lungs, pancreas, and bone marrow as a measure of the immunomodulatory ability of the substance.

In one embodiment dendritic cells, T cells, T regulatory, B cells or macrophages can be purified or differentiated from mice where expression of IL-10 (or Foxp3 in the case of T cell analysis) is marked by the fluorophore Green fluorescent protein (GFP). In those embodiments, the candidate bacterial substance is contacted with one of the aforementioned cell types and the amount of GFP expression will be determined indicating the immunomodulatory ability of the substance being tested. Alternatively, dendritic cells or other antigen presenting cells can be incubated with the substance, the substance can be washed off and the dendritic cells can be subsequently incubated with T cells to determine the ability of the dendritic cells to elicit immunomodulatory activity from the T cell. GFP expression can be determined using fluorescent cell sorting (FACS) or a microplate reader.

In an embodiment, a T cell, antigen presenting cell, and reagents for detection of at least one of one or more anti-inflammatory biomarkers selected from the group consisting of IL-10, Foxp3, TGFβ2, Perforin and Granzyme B and one or more labeled inflammatory biomarker selected from the group consisting of IL17 and TNFα, an be comprised in a system for identifying a bacterial substance having immunomodulatory ability according to methods herein described.

In an embodiment, the system comprises: dendritic cell, T cell, T regulatory, B cells and/or macrophages that, in some embodiments, can be purified or differentiated from mice where expression of IL-10 (or Foxp3 in the case of T cell analysis) is marked by the fluorophore Green fluorescent protein (GFP).

The systems can be provided in the form of kits of parts. In a kit of parts, the multi-ligand capture agent and other reagents to perform the method can be comprised in the kit independently. The antigen presenting cell, the T cell and the reagents can be included in one or more compositions, and each cell and reagent can be in a composition together with a suitable vehicle.

Additional components can include labeled molecules and in particular, labeled capture agents specific for an anti-inflammatory or an inflammatory biomarker or a molecule associated to the expression thereof, a microfluidic chip, reference standards, and additional components identifiable by a skilled person upon reading of the present disclosure.

The term “capture agent” as used herein indicates a compound that can specifically bind to a target. The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. By “stable complex” is meant a complex that is detectable and does not require any arbitrary level of stability, although greater stability is generally preferred. In some embodiments, the kit can comprise labeled polynucleotides or labeled antibodies.

The components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further details concerning the identification of the suitable carrier agent or auxiliary agent of the compositions, and generally manufacturing and packaging of the kit, can be identified by the person skilled in the art upon reading of the present disclosure.

### EXAMPLES

The methods and systems herein described and the related compositions are further illustrated in the following examples, which are provided by way of illustration and are not intended to be limiting.

In particular, the following examples illustrate exemplary cell cultures, methods for contacting PSA with cells and detecting expression of IL-10, Foxp3, TGFβ1, TGFβ2, perform, granzyme B, TNFα IL17A, A person skilled in the art will appreciate the applicability of the features described in detail for PSA and Bacteroides Fragilis and cells/cultures/animal systems suitable for detecting PSA and Bacteroides fragilis immunomodulatory activity for additional substances according to the present disclosure. Similarly a skilled person will appreciate the applicability of the detection of a biomarker such asIL10, Foxp3, TGFβ1, TGFβ2, perform, granzyme B, TNFα IL17A to other biomarkers herein described.

The following material and methods were used for all the methods and systems for detection of immunomodulatory substances exemplified herein.

Bacterial strains and culture conditions and mice B. fragilis strain NCTC 9343 was obtained from the American Type Culture Collection, its isogenic PSA deletion mutant and mpi44 mutant (produces only PSA but not other polysaccharides) has been described (M. J. Coyne, A. O. Tzianabos, B. C. Mallory, V. J. Carey, D. L. Kasper and L. E. Comstock, (2001) Polysaccharide biosynthesis locus required for virulence of Bacteroides fragilis, Infect. Immun. 69: 4342-4350.) Bacteria were grown either in a rich medium containing 37 g BHI (BD #237200), 0.5 μg/ml Hemin (Sigma H5533), and 0.5 μg/ml Vitamin K (Sigma V3501) in 1 L ddH₂O or a customized minimum medium (MM), which contained 8 g Glucose, 1% FBS, 0.5 μg/ml Hemin, and 0.5 μg/ml Vitamin K in 1 L of RPMI (Invitrogen SKU#11835-030). SPF mice of strains C57BL/6 and Balb/c were purchased from Taconic Farms (Germantown, N.Y.). TLR2^{-/-}and IL-10 knockout mice were purchased from Jacksom laboratories IL-10GFP mice were procured from the laboratory of Christopher Karp from Cinncinati Childrens medical hospital, and, Foxp3GFP mice were a given by the laboratory of Talal Chatila from the University of California Los Angeles.

Isolation of EDL-enriched bacterial population. Percoll (GE Healthcare #17-0891-01) discontinuous density-gradient centrifugation was used for EDL isolation of both wild-type B. fragilis and B. fragilis ΔPSA (Patrick S, Reid J H. (1983) Separation of capsulate and non-capsulate Bacteroides fragilis on a discontinuous density gradient. J Med. Microbiol. 16(2): 239-41.) Briefly, a 20%, 40%, 60%, 80% Percoll gradient (diluted with PBS) was created in a 14 ml test tube (2 ml for each layer). Then B. fragilis culture resuspended in PBS was carefully added on top of the 20% Percoll layer. Subsequently, the gradient was centrifuged at 800 g for 20 min at RT. EDL-enriched bacteria can be recovered from the 40%-60% interface of the gradient after the separation.

OMV purification and labeling. This method is adapted from a previously described protocol for the preparation of OMVs from E. coli (Amanda L. Horstman and Meta J. Kuehn. (2000) Enterotoxigenic Escherichia coli secretes active heat-labile enterotoxin via outer membrane vesicles. J Biol. Chem. 275: 12489-12496.) Briefly, EDL-enriched B. fragilis was grown in customized MM. OMVs were recovered from the bacteria-free supernatant of the culture by centrifugation at 40,000 g for 2 hrs at 4° C. and further washed twice with PBS and filtered through 0.45 μm spin columns (Millipore #20-218). Total protein concentration of the purified OMVs was determined by Bradford assay (Biorad #500-0205). FITC-labeled OMVs were prepared as previously described (Nicole C. Kesty and Meta J. Keuhn. (2004) Incorporation of heterologous outer membrane and periplasmic proteins into Escherichia coli outer membrane vesicles. J Biol. Chem. 279: 2069-2076). Briefly, OMVs were incubated in the staining buffer (1 mg/ml FITC (Thermo Scientific #46424), 100 mM NaCl, 50 mM Na₂CO₃, pH 9.2) for 1 hr at RT. Labeled OMVs were collected by centrifugation at 40,000 g for 30 min at 4° C. and washed twice with PBS+200 mM NaCl.

Electron microscopy of bacterial ultrathin section. Ultrathin sections of EDL-enriched B. fragilis were prepared as previously described (Patrick S, McKenna JP, O'Hagan S, Dermott E. (1996) A comparison of the haemagglutinating and enzymic activities of Bacteroides fragilis whole cells and outer membrane vesicles. Microb Pathog. 20(4):191-202.) Briefly, samples were fixed in 2.5% (v/v) glutaraldehyde (Sigma, G5882) in cacodylate buffer overnight at 4° C., followed by further fixation in osmium tetroxide (1%, w/v) for 3 hrs at RT in the dark. Ruthenium Red (1 mg/ml, Sigma R2751) was included in both of the fixation processes. Then fixed samples were embedded in epoxy resin after dehydration in a graded series of alcohols. Ultrathin sections (100-200 nm) were cut and negatively stained with 2% uranyl acetate and lead citrate on formvar/carbon coated copper grids (EMS #FCF200-Cu) before visualization by TEM.

Immunogold labeling of purified OMVs. This method was adapted from a previously described protocol (Patrick S, McKenna JP, O'Hagan S, Dermott E. (1996) A comparison of the haemagglutinating and enzymic activities of Bacteroides fragilis whole cells and outer membrane vesicles. Microb Pathog, 20(4):191-202.) Briefly, a tiny drop of purified OMV was applied to formvar/carbon coated gold grids (EMS #FF200-Au) and air-dried. Immunogold labeling was performed at RT by floating these grids with “OMVs”-side down on a series of small drops of antibody and wash solutions. Particularly, samples were blocked in 10% FBS for 10 min after 5 min incubation in 0.12% glycine. After blocking, samples were further incubated with antibody against PSA diluted in 10% FBS for 20 min, followed by 5 washes×3 min with PBS. Subsequently, secondary antibody-IgG conjugated with 5 nm gold (kind gift from Dr. Paul Webster, House Ear Institute, Los Angeles) was applied to the samples for 20 min, followed again by 5 washes×4 min with PBS. After labeling, samples were fixed in 1% glutaraldehyde for 5 min and washed extensively by transferring grids to drops of PBS 4×1 min and H₂O 4×1 min. Contrast staining was performed by placing the grids on drops of 3-5% uranyl acetate in 2% methylcellulose for 10 min on ice. Finally, grids were removed from the staining solution by a wire loop and air-dried. Samples covered by a thin film of methylcellulose were removed from loop and used for visualization by transmission electron microscopy (TEM).

Glycoprotein assay. PSA purified from whole cell extracts or OMVs (from B. fragilis mutant mpi44) were subjected to SDS-PAGE and the gel was stained subsequently by glycoprotein staining kit (G bioscience #786-254) to show the presence of PSA.

Chemical (TNBS)-induced experimental colitis. This protocol is adapted from a previously described method (Scheiffele and Fuss. (2001) Induction of TNBS colitis in mice. Current Protocols in Immunology. 15.19.1-15.19.14.) Briefly, wild-type (Balb/c) male mice were orally treated with PBS, WT-OMV (5 μg) or ΔPSA-OMV (5 μg) every other day for a week before TNBS administration. The treated mice were anesthetized with isofluorene and rectal administration of 2% TNBS (in 50% EtOH, Sigma P2297) was applied through a 3.5 F catheter (Instech Solomon; SIL-C35). Oral treatment continued for another two times after TNBS administration and mice were analyzed 1-2 days after the last treatment.

Tissue pathology analysis Mouse colons were fixed in neutral buffered 10% formalin (ScyTek Laboratories CAS#50-00-0) and processed by Pacific Pathology for H & E staining. Colitis scores for each colon section were evaluated in a blinded fashion by a pathologist (Dr. Gregory Lawson, David Geffen School of Medicine, UCLA, Los Angeles). Histology images were taken using light microscopy (Zeiss) at 20× magnification.

Quantitative real-time PCR RNA was collected either from mouse tissues using Trizol (Invitrogen #15596-018) or from purified cells using RNeasy Mini Kit (Qiagen #74104). iSCRIPT cDNA synthesis kit (BioRad #170-8890) was used for conversion of cDNA and IQ SYBR Green supermix (BioRad #172-8882) was used for real-time PCR. Primers used in this study are: TNFα (F-5′ACG GCA TGG ATC TCA AAG AC 3′ (SEQ ID NO:1); R-5′ GTG GGT GAG GAG CAC GTA GT 3′) (SEQ ID NO 2); IL-17 (F-5′ TTA AGG TTC TCT CCT CTG AA 3′(SEQ ID NO:3); R-5′ TAG GGA GCT AAA TTA TCC AA 3′) (SEQ ID NO: 4) IL-10 (F-5′ GGT TGC CAA GCC TTA TCG GA 3′ (SEQ ID NO:5); R-5′ ACC TGC TCC ACT GCT TGC T 3′) (SEQ ID NO: 6) Foxp3 (F-5′ GCA ATA GTT CCT TCC CAG AGT TCT 3′(SEQ ID NO: 7); R-5′ GGA TGG CCC ATC GGA TAA G 3′ (SEQ ID NO: 8)).

Fluorescent microscopy In vitro differentiated BMDCs were plated into Lab-Tek II S-well chamber slide (Nunc #154534) at 50,000 cells/well. FITC labeled OMVs were added into the cell culture at 10 μg/ml. After 2 hrs incubation, cells were fixed in 4% PFA for 20 min at RT. After 3 washes×5 min with PBS, cell membrane was stained with 1 μg/ml tetramethylrhodamine conjugate of WGA (Invitrogen W849) for 1 hr at 4° C. And ProLong Gold anti-fade reagent (P36930) was applied to the sample after extensive washes following membrane staining. Fluorescent images were taken using LSM 510 microscope and Plan-Neofluar 63×/1.25 oil objective.

Flow cytometry and staining BMDCs from OMV uptake assay or activation assay were collected and blocked in 5% mouse serum for 30 min on ice. After blocking, cells were stained with anti-CD11c-APC, anti-MHCII-FITC or anti-CD86-PE (ebioscience) for 30 min on ice and washed 2× with FACS buffer (HBSS (no Ca²⁺/Mg²⁺), 1% FBS, 2 mM EDTA, 10 mM HEPES) at 4° C. before flow cytometry analysis. Similarly, cells from in vitro BMDC-T cell co-culture were blocked, and stained with anti-CD4-APC/anti-CD25-PE the same way except that the cells were re-stimulated using PMA/Ionomycin for 4-4.5 hrs before collecting. All flow cytometry was done with BD FACSCalibur and results were analyzed using FlowJo.

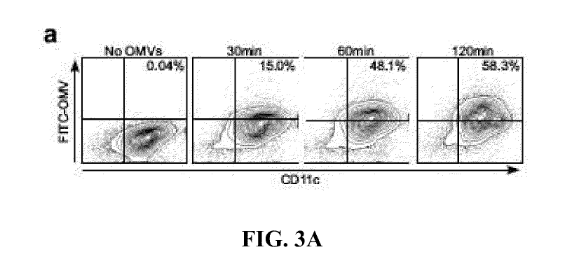

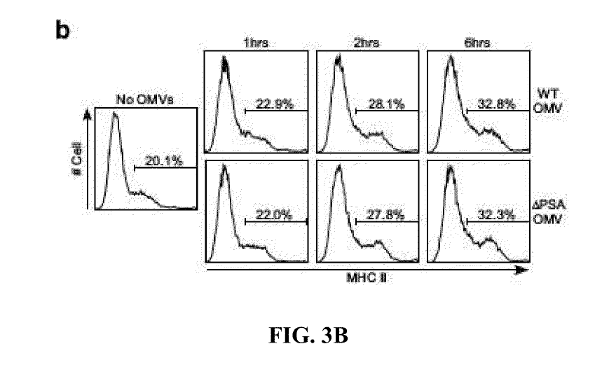

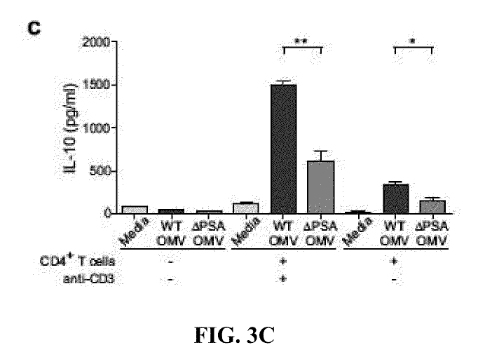

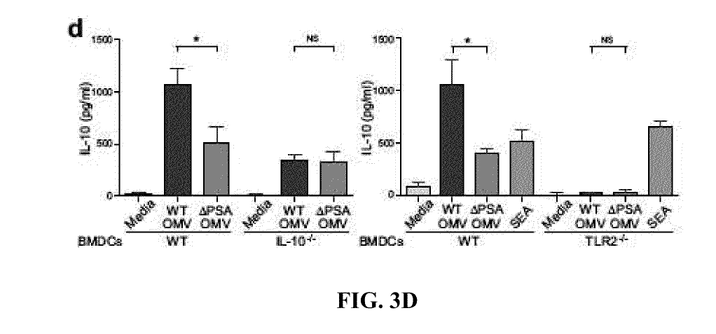

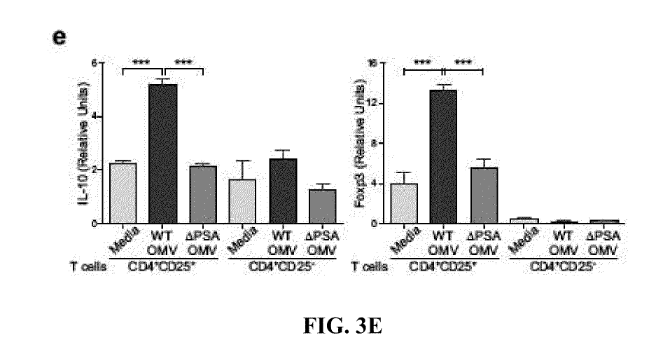

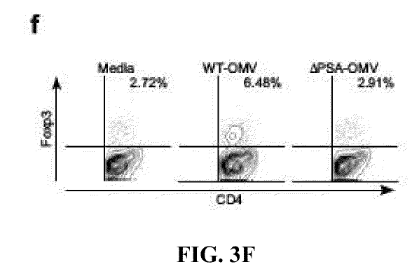

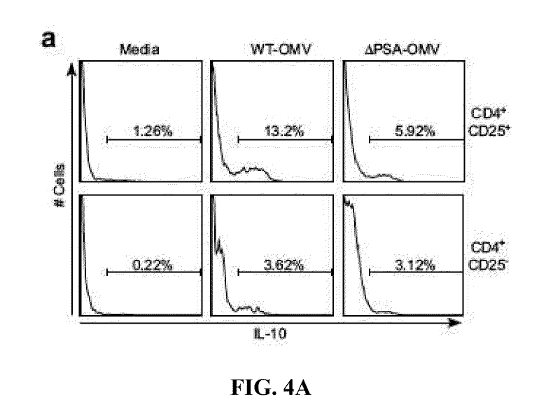

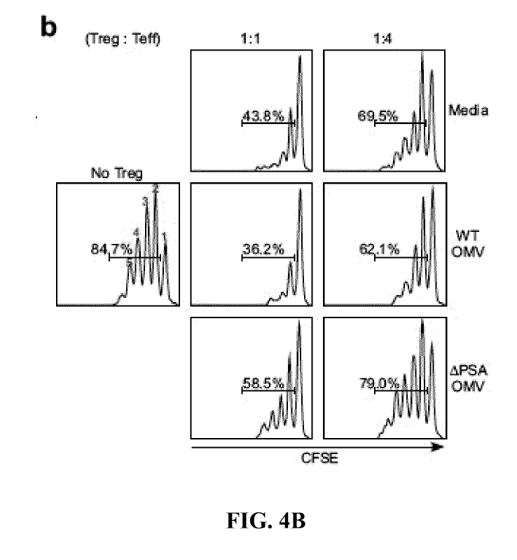

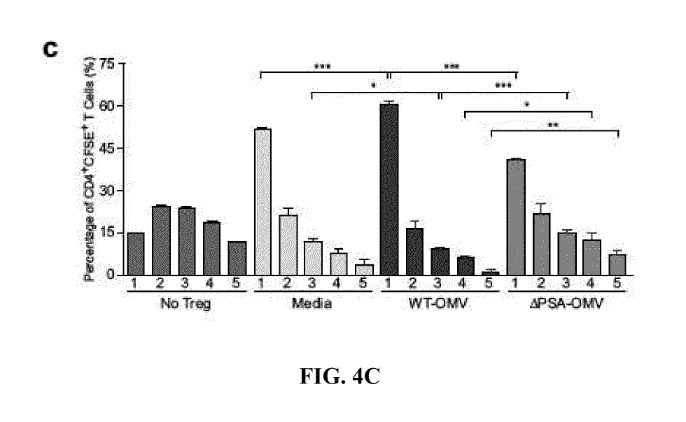

In vitro BMDC-T cell co-culture. Bone marrow was collected from different strains of mice and differentiated in vitro in the presence of 20 ng/ml GM-CSF (Miltenyi Biotec #9517571) for 8 days as described previously (Mazmanian, Liu, Tzianabos, and Kasper (2005) An Immunomodulatory Molecule of Symbiotic Bacteria Directs Maturation of the Host Immune System. Cell 122:1 107-118.). (Cell purity was >90%.) CD4⁺ splenic T cells were isolated by magnetic microbead purification (Miltenyi Biotec #130-090-860) (Cell Purity is >95%.) OMV-pulsed BMDCs (100 μl/ml OMVs, 100,000 cells/ml, 12 hrs⁻²⁴ hrs) were washed with HBSS and then incubated with CD4⁺T cells (1,000,000 cells/ml) in a round bottom 96 well plate with addition of 0.01 μl/ml anti-CD3 (day 0, FIG. 3c as indicated, 3d, e, f, FIG. 4a), 2 ng/ml TGFb (day 0, FIG. 3c, d & f, FIG. 4a), and 5 ng/ml IL-2 (day 1 and day 3, all in vitro DC-T cell co-culture assays). After total 4 days of culture, supernatants were collected for ELISA (ebioscience #88-7104-77) or cells were harvested for staining and flow cytometry analysis.

In vitro suppression assay: CD4⁺CD25⁺ cells purified from BMDC (pulsed with WT-OMV or ΔPSA-OMV)-T cell co-culture were used as a source of Tregs (Miltenyi Biotec, #130-091-041). CD4 depleted mouse splenocytes treated with Mitomycin C (Sigma M4278) were used as APCs (100,000 cells/ml). CD4⁺CD25⁻T cells directly purified from mouse spleen were pulsed with CFSE for 10 min at 37° C., followed by first wash with PBS and a second wash with culture media, and used immediately (500,000 cells/ml) as responder cells (Teff). This assay was conducted in a round bottom 96 well plate with an addition of 5 μg/ml of anti-CD3 (ebioscience #16-0031-86) in 2000 volume. Teff:Treg ratio was titrated and cells were collected after 2-3 days of culture for FACS analysis.

Statistical Analyses.: Student T test and one-way ANOVA were applied for pair-wise comparisons and comparisons among >2 groups, respectively. Significant differences among groups detected by ANOVA were analyzed using Newman-Keuls test as the post-hoc test to identify groups exhibiting statistically significant differences. All error bars indicate SEM. NS: not significant; * p<0.05; ***p<0.01; ***p<0.001.

### Example 1

### Immunomodulatory Capsular Polysaccharide PSA is Actively Sorted Into OMVS of B. fragilis.

Ultrathin sections of EDL-enriched B. fragilis were prepared as described in materials and methods and imaged by transmission electron microscopy.

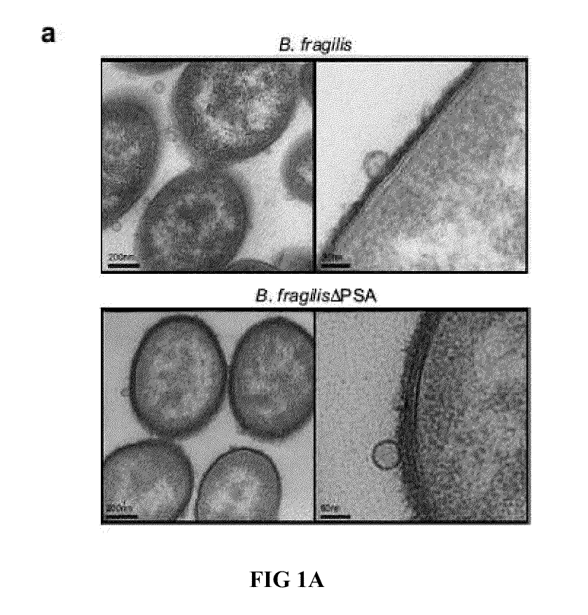

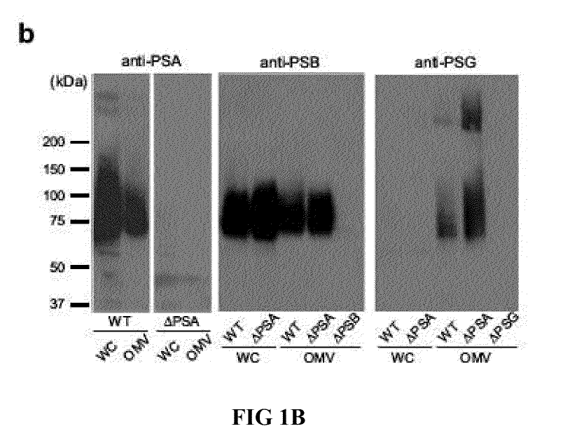

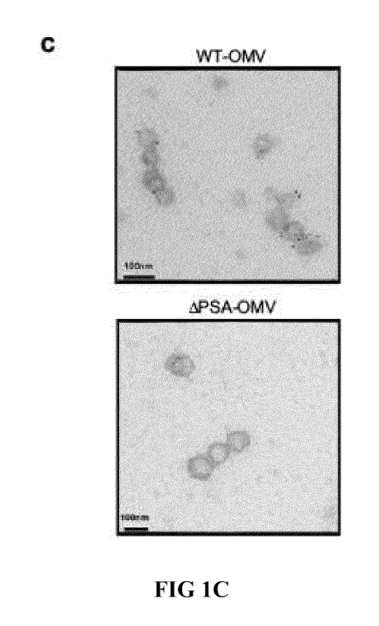

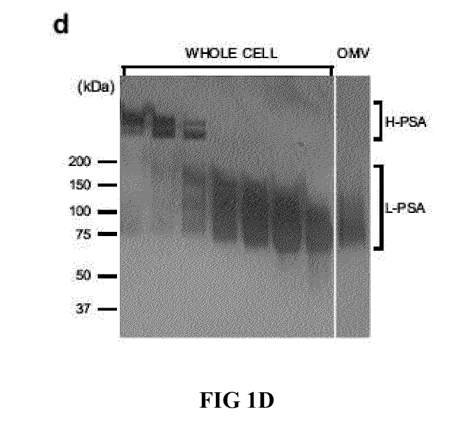

The results illustrated in FIG. 1a show that OMVs were abundantly produced by bacteria, and could be observed budding from the bacterial envelope (FIG. 1a, higher magnification). Applicants' previous studies have shown that deletion of PSA abrogates the immunomodulatory capacity of B. fragilis (Mazmanian, Liu, Tzianabos, and Kasper (2005) An Immunomodulatory Molecule of Symbiotic Bacteria Directs Maturation of the Host Immune System. Cell 122:1 107-118.) (Mazmanian, Round, and Kasper (2008) A microbial symbiosis factor prevents intestinal inflammatory disease. Nature. 453 (7195) 620-625. Electron micrographs of a PSA mutant strain (B. fragilisΔPSA) illustrate no defect in OMV synthesis, and the size, shape and abundance of OMVs produced were indistinguishable from wild-type bacteria (FIG. 1a and FIG. 5). In particular, the results illustrated in FIG. 1a reveal that vesicles are actively budding from the surface of bacteria.

To determine if PSA is associated with OMVs of B. fragilis, purified vesicles from wild-type and ΔPSA bacteria were subjected to immunoblot analysis as described in the materials and methods section.

The results illustrated in FIG. 1b show that the vesicles from wild-type displayed immunoreactivity for PSA, unlike OMVs from B. fragilisΔPSA. B. fragilis produces at least 8 distinct capsular polysaccharides which coat the surface of bacterial cells, named PSA-PSH. While PSB was also detected in vesicle preparations, PSG was absent, demonstrating selectivity for certain polysaccharides to be packaged with OMVs (FIG. 1b). Accordingly, the results of FIG. 1b show that PSA and PSB are associated with vesicles, while PSG is only found on the bacterial surface. Deletion mutants for capsular polysaccharides confirm specificity of each anti-serum.

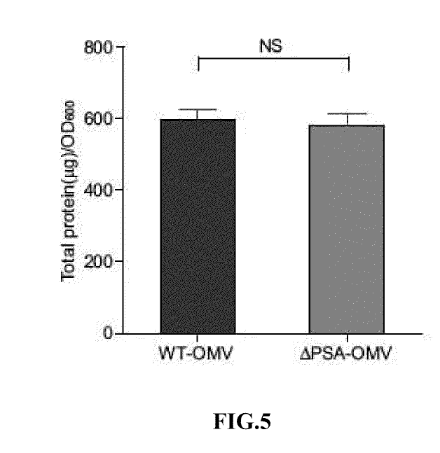

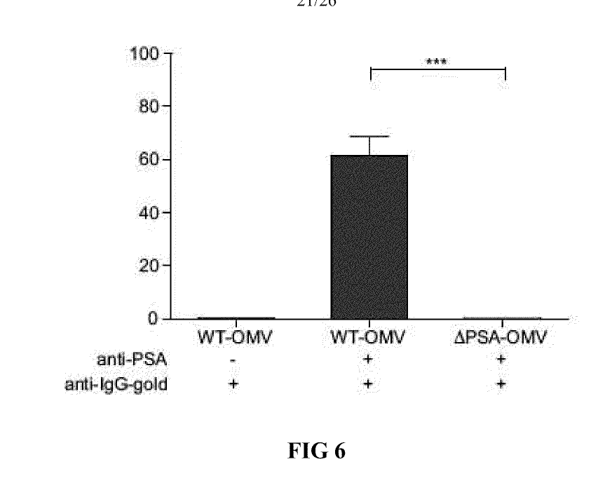

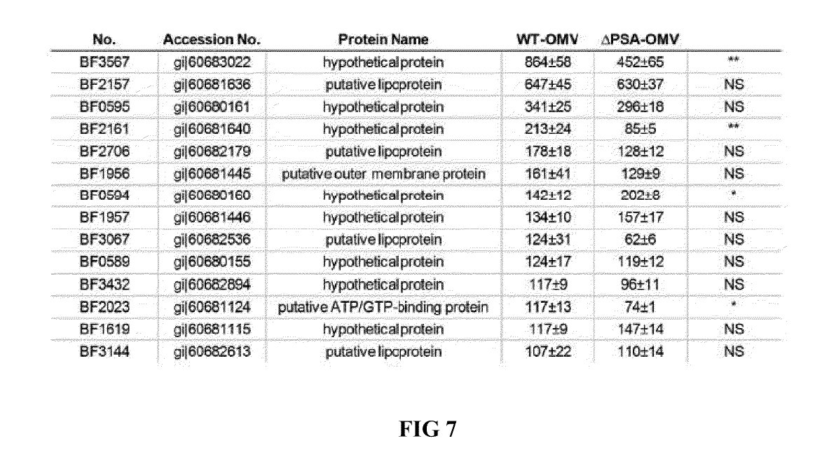

The results from immunoblot analysis were confirmed by experiments of immunogold labeling performed as described in the materials and methods section. The results of immunogold labeling of purified vesicles illustrated in FIG. 1c and confirm that PSA is physically associated with OMVs, and that the vast majority of OMVs from wild-type B. fragilis stain positively for PSA (FIG. 6). To verify that the absence of PSA did not alter the molecular composition of OMVs, a proteomic analysis was performed by mass spectrometry which revealed no qualitative or quantitative differences in the protein composition between vesicles from wild-type or PSA-mutant bacteria (FIG. 7).

PSA is a heterogeneous polymer of repeating subunits. Size separation of PSA recovered from whole cell extracts by chromatography was performed as well as an immunoblot analysis with anti-PSA of capsular polysaccharide preparations from whole cells and purified OMVs as indicated in material and methods.

The relevant results illustrated in FIG. 1d surprisingly show that only the low molecular weight species is associated with OMVs, illustrating specificity of PSA packaging into vesicles. In particular, the results of FIG. 1d show that only low molecular weight PSA (L-PSA) is packaged into vesicles unlike the high molecular weight (H—PSA) species that remains associated with the bacterial cell envelope.

Together, the above results reveal that the immunomodulatory capsular polysaccharide

PSA is actively sorted into OMVs of B. fragilis.

### Example 2

### OMVS Protect Animals from Experimental Colitis and Intestinal Inflammation in a PSA-Dependent Manner

To investigate if OMVs can ameliorate clinical symptoms of disease, mice were treated by gavage with OMVs during the induction of TNBS (2,4,6-trinitrobenzene sulfonic acid) colitis.

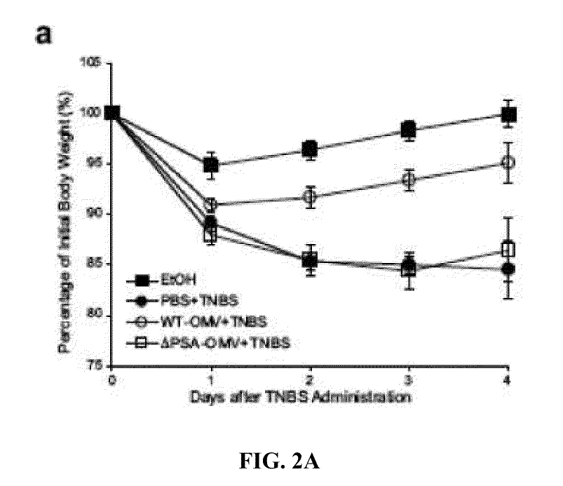

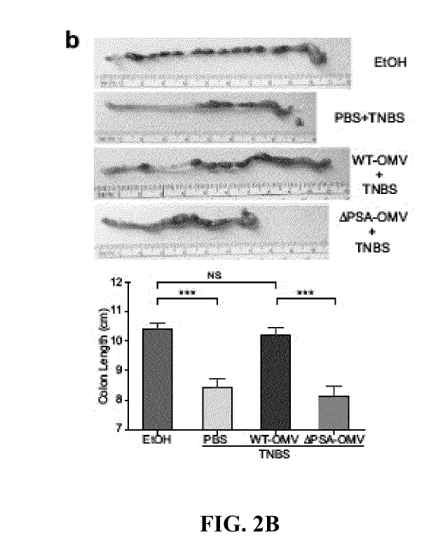

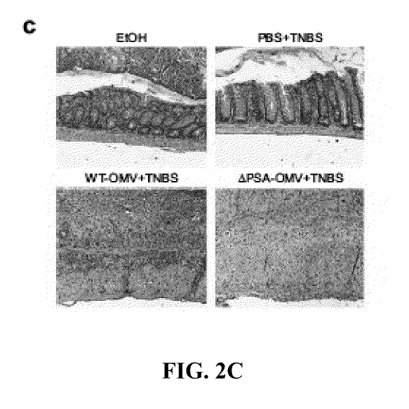

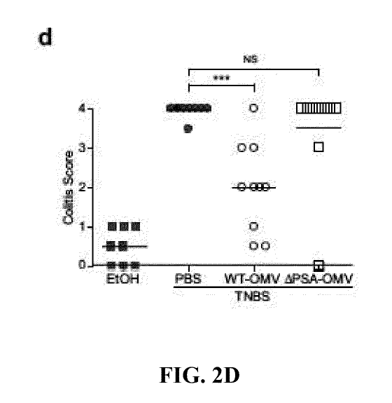

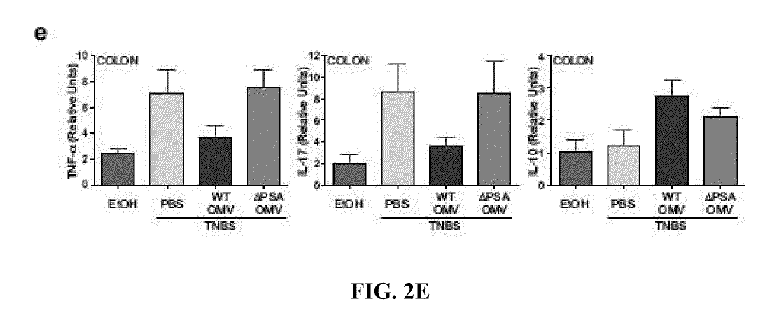

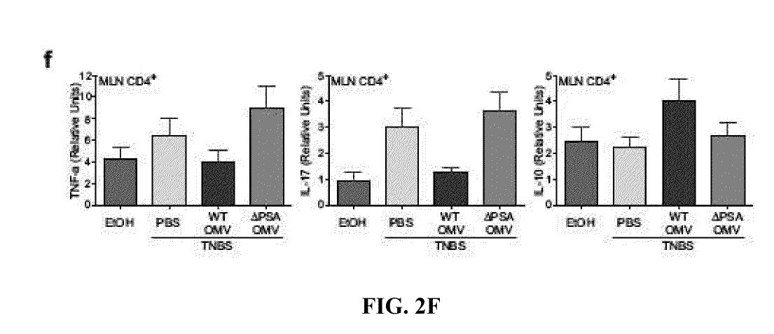

The results illustrated in FIG. 2a indicate that control animals rapidly lost weight following rectal administration of TNBS (FIG. 2a; TNBS+PBS), which did not recover compared to vehicle treated mice (FIG. 2a; ETOH+PBS). Remarkably, OMVs given orally to TNBS animals significantly protected from weight loss (FIG. 2a; TNBS+WT-OMV). Most importantly, when OMVs from B. fragilisΔPSA were administered, weight loss was indistinguishable from TNBS animals (FIG. 2a; TNBS+ΔPSA-OMV), demonstrating that PSA is entirely responsible for preventing wasting disease.

Our efforts to detect intact vesicles in the colon after intra-gastric gavage were confounded by observations of host-derived vesicles, even in germ-free (sterile) mice as previously reported (data not shown).

Since reduction in colon length is a hallmark of TNBS colitis detection of colon length was performed in unmanipulated colons immediately following resection from cecum to rectum and quantification of length (graph) from vehicle treated (EtOH) and TNBS groups (n=4 animals/group). In particular, measurement were performed at time of sacrifice (day 4 following disease induction).

The results illustrated in FIG. 2b showed normal intestinal lengths in animals treated with PSA-containing vesicles, but not in animal trated with OMVs deficient in PSA (FIG. 2b).

Experimental colitis results in severe pathological alterations in intestinal architecture. Accordingly, hematoxylin and eosin (H & E) stained colon sections representative of each treatment group were provided and are shown in FIG. 2c. The images of FIG. 2c show that upon histological analysis of colonic tissues, a significant disease was observed in TNBS treated animals that were ameliorated by oral administration of PSA-containing vesicles.

The results of FIG. 2c were confirmed by colitis scores from animals assigned by a blinded pathologist TNBS colitis manifests itself in focal lesions throughout the colon which mimic the pathology observed in Crohn's disease. To quantitatively assess disease, clinical symptoms were evaluated by a blinded pathologist using a standard scoring system (Scheiffele and Fuss. (2001) Induction of TNBS colitis in mice. Current Protocols in Immunology. 15.19.1-15.19.14.) The results illustrated in FIG. 2d, indicate that while all TNBS and ΔPSA-OMV treated animals were severely affected, WT-OMVs significantly reduced disease in most animals. Animals treated with OMVs from wild-type bacteria had much fewer focal sights of pathology, and when lesions were observed, they were smaller and retained considerably normal tissue architecture compared to animals given ΔPSA-OMV.

The above results establish that PSA is required for the disease protective activity of OMVs.

### Example 3

### PSA Containing OMVS Inhibits TNF-α/IL-17, Enhances IL-10 Expression

The production of canonical pro- and anti-inflammatory cytokines associated with colitis was measured in mice treated as exemplified in Example 2. In particular, cytokine transcript analysis was performed by qRT-PCR from RNA recovered from whole colons or purified CD4⁺T cell from mesenteric lymph nodes.

The relevant results representative of 3 independent trials are reported in FIG. 2e (whole colons) and FIG. 2f (purified CD4⁺T cell from mesenteric lymph nodes). Those results show that transcript levels of tumor necrosis factor (TNF-α and IL-17A were elevated in TNBS-treated animals, but reduced by OMV administration in a PSA-dependent manner (FIG. 2e). Consistent with protection from pathology, OMVs elicited the production of increased IL-10 levels compared to animals orally given ΔPSA-OMV (FIG. 2f). Analysis of cytokine production by purified CD4⁺T cells from mesenteric lymph nodes (MLNs) confirmed that IL-10 was indeed being produced by T cells in response to PSA (FIG. 2f). Infiltration of Th17 cells, which are required for disease, were significantly reduced by OMVs, as were levels of the potent pro-inflammatory marker TNF-α□ (FIG. 2f). We conclude that PSA packaging into OMVs of B. fragilis protects animals from the pathological and immunological manifestations of experimental colitis.

### Example 4

### PSA Containing OMVS from B. Fragilis Induce Dendritic Cell Responses

Dendritic cells (DCs) extend protrusions into the gut lumen and sample intestinal particles, and subsequently migrate to MLNs in order to initiate T cell reactions. Indeed, PSA administered orally to animals is associated with CD11c⁺DCs in the MLN. Accordingly, Applicants sought to test whether OMV containing PSA can also be taken up by DCs.

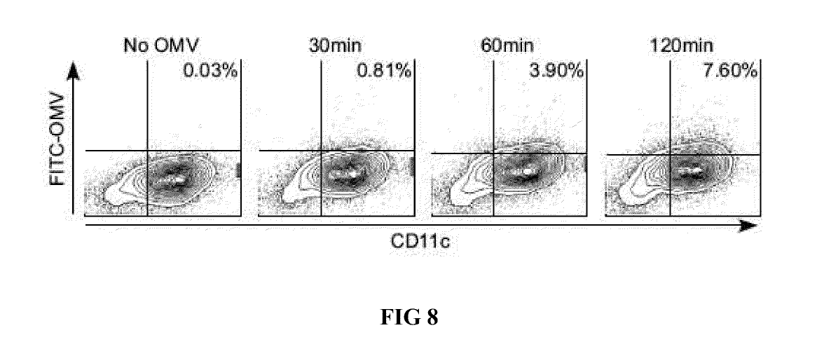

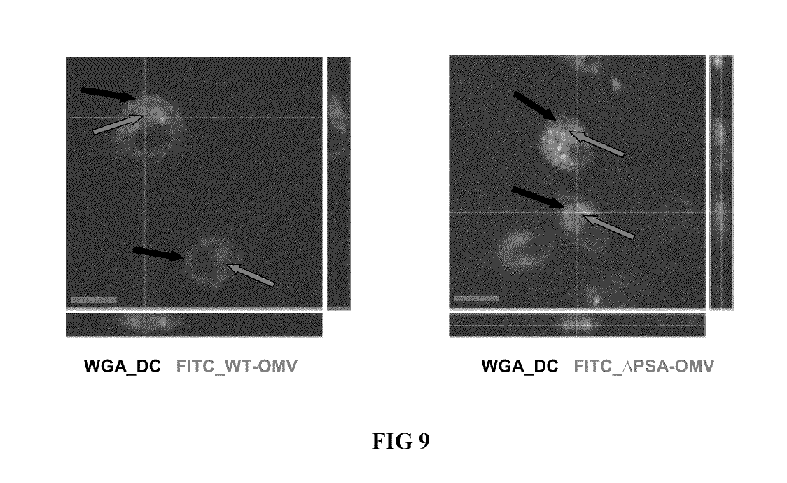

The results illustrated in FIG. 3a show that bone-marrow derived DCs rapidly internalized OMVs in an actin-dependent manner as treatment of cells with cytochalsin D significantly inhibited vesicle uptake (FIG. 3a and FIG. 8). Intercellular localization was confirmed by confocal microscopy (FIG. 9).

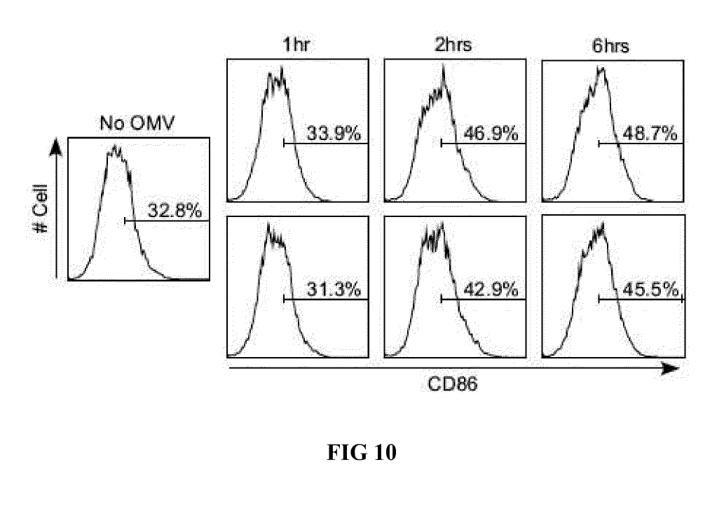

PSA mediated induction in the DCs of T cell activation markers was also investigated. The results illustrated in FIG. 3b indicated that expression of T cell activation markers (MHCII, CD86) was elevated equally following internalization of both WT-OMVs and ΔPSA-OMVs (FIG. 3b and FIG. 10).

Increased expression of MHC and co-stimulatory molecules indicate that PSA containing OMVs from B. fragilis can influence T cell responses.

### Example 5

### PSA Induce IL-10 Expression in CD4⁺T Cell Through DCS IL-10 Expression

In light of PSA's protective role in colitis shown with experiments exemplified in Examples 1 to 3, the biological effects of OMVs on the induction of suppressive T cell responses were tested. In particular, as various regulatory T cell (Treg) populations are known to inhibit colitis, OMVs' ability to promote Treg development was examined.

The results illustrated in FIG. 3c indicate that WT-OMVs induced the expression of IL-10 from in vitro DC-T cell co-cultures, while no IL-10 was produced from DCs alone under these conditions. Vesicles purified from B. fragilisΔPSA induced significantly less IL-10 than WT-OMVs, although an increase was detected over media controls. The production of IL-10 from DCs is known to support CD4⁺IL-10⁺T cell development in vivo and in vitro. Accordingly, ELISA analysis similar to the one whose results are illustrated in FIG. 3c, was performed also including DCs differentiated from IL-10^{-/-}animals.

The results illustrated in FIG. 3d (left panel) show a greatly reduced IL-10 production in DC-T cell co-cultures when IL-10 DCs were treated with OMVs, suggesting IL-10 expression by DCs is required to induce IL-10 from CD4⁺T cell in a paracrine manner.

### Example 6

### PSA Programs DCs to Direct Foxp3 Treg Development and/or Expansion

Microbial ligands are sensed by several classes of pattern recognition receptors, and PSA has been shown to signal through toll-like receptor 2 (TLR2) to elicit Th1 cytokine production. A series of experiments were therefore performed to test whether TLR2 is required for induction of IL-10 by PSA, as recent reports have shown that Treg function and IL-10 expression are influenced by TLR2.

The results illustrated in FIG. 3d (right panel) indicate that compared to wild-type DCs, the absence of TLR2 (DCs from TLR2^{-/-}animals) completely inhibit IL-10 production in response to OMVs. Both DCs responded equally to superantigen (SEA) stimulation, demonstrating a specific defect in PSA sensing and not a general lack of T cell activation by TLR2^{-/-}DCs (FIG. 3d). CD4⁺CD25⁺T cells that express the transcription factor Foxp3 are an important Treg subset. Recent studies have shown that CD4⁺CD25⁺Foxp3⁺Tregs can express IL-10, and IL-10 production from Tregs is required to prevent intestinal inflammation.

To determine the source of IL-10 induced by PSA on OMVs, CD4⁺CD25⁺ and CD4⁺CD25⁻T cells were purified following co-culture with DCs and the expression of IL-10 and Foxp3 were measured by qRT-PCR.

The results illustrated in FIG. 3e indicate that remarkably, PSA significantly induced IL-10 expression in the CD4⁺CD25⁺Treg population, but not from CD4⁺CD25⁻T cells. OMVs purified from B. fragilisΔPSA were unable to promote IL-10 production from either T cell population, with levels identical to media controls. Expression of Foxp3 was also significantly increased exclusively in CD4⁺CD25⁺T cells by OMVs in a PSA-dependent manner (FIG. 3e).

co-cultures were set up as for experiments illustrated in FIGS. 3c-e, but using CD4⁺T cells from Foxp3-GFP mice. Following 4 days of culture with OMV pulsed DCs, cells were stained with anti-CD4 and Foxp3 detected by GFP expression using FC.

The results representative of 2 independent trials are illustrate in FIG. 3f. Consistent with the increased transcription of Foxp3 among CD4⁺T cells, proportions of CD4⁺Foxp3⁺T cells increased in response to WT-OMV but not ΔPSA-OMV treatment (FIG. 3f). Collectively, OMVs containing PSA program DCs to direct Treg development and/or expansion in an entirely in vitro culture system.

### Example 7

### PSA-Containing OMVS Induce IL10 Mediated Foxp3 Treg Suppressive Function

The use of Tregs as cellular therapies has been proposed for IBD, autoimmunity and allergies. IL-10 expression by CD4⁺T cell subsets following 4 day co-culture with DCs treated with OMVs was investigated. Splenic CD4⁺T cell were purified from IL-10-GFP mice, stained with anti-CD4 and anti-CD25 following co-culture, and IL-10 expression measured by GFP expression.

The results of the relevant OMV treatment of purified DCs & T cells illustrate in FIG. 4a indicate that revealed that PSA promotes the specific production of IL-10 from CD4⁺CD25⁺Tregs.

Based on this finding, the ability of PSA was investigated to promote the suppressive capacity of Tregs ex vivo. in vitro suppression of naïve responder cells by purified CD4⁺CD25⁺T cells following co-culture with DCs treated with media (control), WT-OMVs and ΔPSA-OMVs. CD4⁺CD25⁻responder cells (effector cells; Teff) were purified from spleens of wild-type mice, pulsed with intracellular dye CFSE (Carboxyfluorescein succinimidyl ester), incubated with Tregs and stimulated with anti-CD3 for 3 days. Cell proliferation was measured by FC of CFSE dilution.

The results illustrated in FIGS. 4b and 4c indicate Tregs recovered from conditions with WT-OMV-treated DCs displayed significantly enhanced suppressive capacity compared to CD4⁺CD25⁺T cells in response to ΔPSA-OMVs.

Therefore, the specific absence of PSA from OMVs that otherwise contain numerous microbial ligands (LPS, lipoproteins, peptidoglycan, etc) abrogates the ability of B. fragilis vesicles to induce functional Tregs.

### Example 8

### PSA Induce Various Biomarkers Combination in Foxp3 Treg

Foxp3-GFP mice were orally treated with purified PSA every other day for 6 days. MLNs were extracted and CD4+Foxp3+ or the CD4+Foxp3−T cells were purified by FACS based on ±GFP expression (purity >99%). RNA was extracted and used for q-PCR.

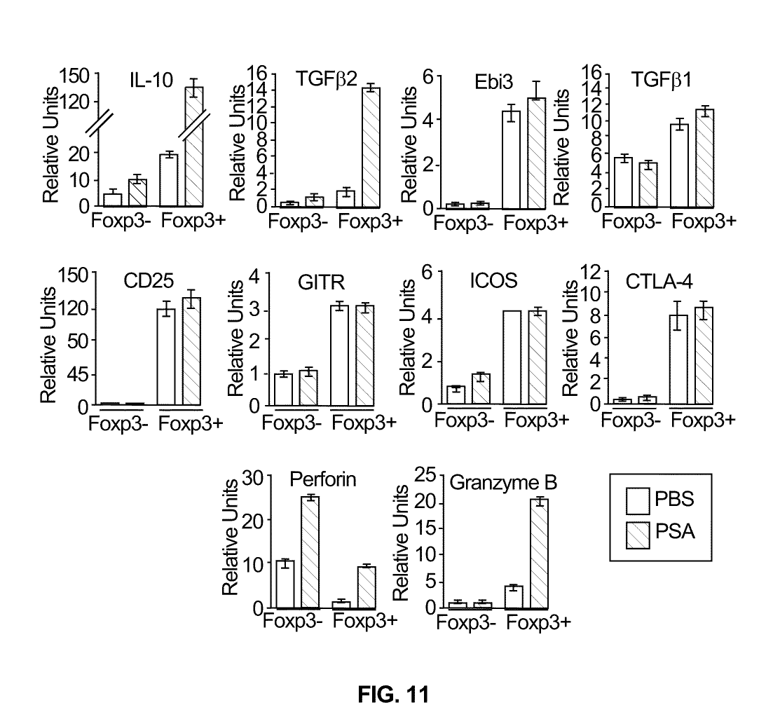

The results illustrated in FIG. 11 demonstrate that PSA coordinates induction of multiple anti-inflammatory genes including IL-10, TGF-β, perforin, and granzyme A and the inhibition of TNFα and IL17 (in particular IL17A).

The above results provide a molecular mechanism for the probiotic activity of B. fragilis, and reveal a seminal example for a microbial ligand that links innate immune receptor signaling to regulatory T cell development. In particular the above results indicate that PSA is delivered to the host by outer membrane vesicles (OMVs), secretion structures that target bacterial molecules to host cells. OMVs containing PSA are internalized by dendritic cells of the host immune system. Following uptake of OMVs, PSA programs dendritic cells to induce the differentiation of regulatory T cells (Treg) that express Foxp3 and the anti-inflammatory cytokine interleukin-10 (IL-10). Treg development by OMVs requires toll-like receptor 2 (TLR2) expression and IL-10 production by dendritic cells. Remarkably, purified OMVs direct the in vitro differentiation of functional Tregs with potent suppressive activity in a PSA dependent manner. Treatment of animals with OMVs containing PSA prevents experimental colitis and suppresses pro-inflammatory cytokine responses in the gut. These findings reveal that commensal bacteria provide beneficial microbial factors through vesicle secretion, a process that can be engineered into a novel approach for delivery of probiotic therapies for IBD.

In particular, the above results show that Tregs induced by OMVs containing PSA are functionally suppressive and inhibit T cell activation in culture. This is the first demonstration of in vitro Treg development by a microbial ligand, and provides proof-of-principle to the widely-speculated notion that innate immune receptor signaling modulates Treg function. As current therapies for IBD are either ineffective or have severe side effects, probiotics represent promising new treatment options by harnessing well-evolved evolutionary mechanisms for immunmodulation. Given that Tregs suppress immune responses in multiple tissues, our seminal demonstration of in vitro Treg development by PSA suggests the exciting possibility of novel cellular therapies for numerous inflammatory disorders including autoimmunity, asthma and allergies.

To identify molecules made by microorganisms that mediate immunomodulation, fractions of bacterial products can be purified and the same assays performed as above until a pure compound is found which mimics that outcome when whole bacteria are used. Also, the approaches above could be used to screen a mutant library of a microorganism of interest to identify immunomodulatory molecules that have been deleted in a respective clone of a strain which possesses this activity.

The examples set forth above are provided to give those of ordinary skill in the art a complete disclosure and description of how to make and use the embodiments of the vesicles, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference. Further, the hard copy of the sequence listing submitted herewith and the corresponding computer readable form are both incorporated herein by reference in their entireties.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the specific examples of appropriate materials and methods are described herein.

A number of embodiments of the disclosure have been described. Nevertheless, it will be understood that various modifications can be made without departing from the spirit and scope of the present disclosure. Accordingly, other embodiments are within the scope of the following claims.

### REFERENCES

1. Xavier, R. J. & Podolsky, D. K. Unravelling the pathogenesis of inflammatory bowel disease. Nature 448, 427-434 (2007).
2. Mazmanian, S. K., Round, J. L. & Kasper, D. L. A microbial symbiosis factor prevents intestinal inflammatory disease. Nature 453, 620-625 (2008).
3. Braun, J. & Wei, B. Body traffic: ecology, genetics, and immunity in inflammatory bowel disease. Annu Rev Pathol 2, 401-429 (2007).
4. Round, J. L. & Mazmanian, S. K. The gut microbiota shapes intestinal immune responses during health and disease. Nat Rev Immunol (2009).
5. Scheiffele and Fuss. (2001) Induction of TNBS colitis in mice. Current Protocols in Immunology. 15.19.1-15.19.14.
6. Mazmanian, Liu, Tzianabos, and Kasper (2005) An Immunomodulatory Molecule of Symbiotic Bacteria Directs Maturation of the Host Immune System. Cell 122:1 107-118
7. M. J. Coyne, A. O. Tzianabos, B. C. Mallory, V. J. Carey, D. L. Kasper and L. E. Comstock, (2001) Polysaccharide biosynthesis locus required for virulence of Bacteroides fragilis, Infect. Immun. 69: 4342-4350.
8. Patrick S, Reid JH. (1983) Separation of capsulate and non-capsulate Bacteriodes fragilis on a discontinuous density gradient. J Med. Microbiol. 16(2): 239-41
9. Amanda L. Horstman and Meta J. Kuehn. (2000) Enterotoxigenic Escherichia coli secretes active heat-labile enterotoxin via outer membrane vesicles. J Biol. Chem. 275: 12489-12496
10. Nicole C. Kesty and Meta J. Keuhn. (2004) Incorporation of heterologous outer membrane and periplasmic proteins into Escherichia coli outer membrane vesicles. J Biol. Chem. 279: 2069-2076.
11. Patrick S, McKenna JP, O'Hagan S, Dermott E. (1996) A comparison of the haemagglutinating and enzymic activities of Bacteroides fragilis whole cells and outer membrane vesicles. Microb Pathog. 20(4):191-202.

What is claimed is:

1. A method for identifying a bacterial substance having immunomodulatory ability, the method comprising
contacting a candidate bacterial substance with a T cell alone or in presence of a cell presenting an antigen;
detecting expression of at least one of one or more anti inflammatory biomarkers selected from the group consisting of IL-10, Foxp3, TGFβ1, TGFβ2, Perforin and Granzyme B, or reducing levels or expression of one or more inflammatory biomarkers selected from the group consisting of IFNγ, IFNα, IFNβ IL-1β, IL-4, IL-5, IL-6, IL-8, IL-9, IL-13, IL-21, IL-22, IL-23, IL-17 and TNFα; and
determining an anti inflammatory ability of the candidate bacterial substance through detection of an increase of the expression of the one or more anti-inflammatory biomarkers or a decrease of the expression of the one or more inflammatory biomarkers following the contacting.

2. A method for identifying a bacterial substance having immunomodulatory ability, the method comprising
contacting a candidate bacterial substance with an antigen presenting cell,
incubating the antigen presenting cell with a T cell following the contacting;
detecting expression of at least one of one or more anti inflammatory biomarker selected from the group consisting of IL-10, Foxp3, TGFβ1, TGFβ2, Perforin and Granzyme B, and
one or more inflammatory biomarker selected from the group consisting of IFNγ, IFNα, IFNβ IL-1β, IL-4, IL-5, IL-6, IL-8, IL-9, IL-13, IL-21, IL-22, IL-23, IL-17 and TNFα; and
determining an anti inflammatory ability of the candidate bacterial substance through detection of an increase of the expression of the one or more anti-inflammatory biomarkers or a decrease of the expression of the one or more inflammatory biomarkers following the incubating

3. The method of claim 1 or 2, wherein the detecting is performed by detecting expression of Foxp3 alone or in combination with IL-10, Foxp3, TGFβ1, TGFβ2, Perforin and/or Granzyme B.

4. The method of claim 1 or 2, wherein the detecting is performed by detecting expression of Foxp3 alone or in combination with IFN7, IFNα, IFNβ IL-1β, IL-4, IL-5, IL-6, IL-8, IL-9, IL-13, IL-21, IL-22, IL-23, IL-17 and/or TNFα.

5. The method of claim 1 or 2, wherein the detecting is performed by detecting expression of Foxp3 and IL-10.

6. The method of claim 1 or 2, wherein the T cell is a T reg cell.

7. The method of claim 1 or 2, wherein the antigen presenting cell is either primary or cell culture derived.

8. The method of claim 1 or 2, wherein the antigen presenting cell is a dendritic cell.

9. The method of claim 1 or 2 wherein the candidate bacterial substance is selected from the group consisting of live bacteria, dead bacteria, bacterial extracts, purified molecules from bacteria, molecules purified from bacteria, and vesicles containing a molecule, or a combination thereof.

10. The method of claim 1 or 2 wherein the candidate bacterial substance comprises outer membrane vesicles.

11. A method for identifying a bacterial substance having immunomodulatory ability in animals, the method comprising
treating a transgenic marker non-human animal with a candidate bacterial substance, the transgenic marker non-human animal genetically modified to express at least one of
one or more labeled inflammatory biomarkers selected from the group consisting of IL-10, Foxp3, TGFβ1, TGFβ2, Perforin and Granzyme B, and
one or more labeled inflammatory biomarker selected from the group consisting of IFNγ, IFNα, IFNβ IL-1β, IL-4, IL-5, IL-6, IL-8, IL-9, IL-13, IL-21, IL-22, IL-23, IL-17 and TNFα; and
detecting expression in the transgenic marker non-human animal of at least one of the one or more anti-inflammatory biomarkers or at least one of the inflammatory biomarkers following the treating and
determining an anti inflammatory ability of the candidate bacterial substance through detection of an increase of the expression of the one or more anti-inflammatory biomarkers or a decrease of the expression of the one or more inflammatory biomarkers following the treating.

12. The method of claim 11, wherein the transgenic non-human animal is a mouse.

13. The method of claim 12 wherein the mouse is selected from the group consisting of an IL-10-GFP mouse and a Foxp3-GFP mouse.

14. The method of any one of claims 11 to 13, wherein the treating is performed by orally or intravenously administering the candidate bacterial substance,

15. The method of any one of claims 11 to 13, wherein the candidate bacterial substance comprises live bacteria and the treating is performed by permanently colonize the transgenic marker non-human mammal with the live bacteria by oral gavage
